(19) United States
(12) Patent Application Publication
(10) Pub. No.: US 2005/0186250 A1
(43) Pub. Date: Aug. 25, 2005

(54) Metallic structures incorporating bioactive materials and methods for creating the same

(75) Inventors: Michael E. Gertner, San Francisco, CA (US); Mordechay Schlesinger, Pittsburgh, PA (US)

Correspondence Address: PRESTON GATES & ELLIS LLP;ATTN: C. RACHAL WINGER, 925 FOURTH AVE, SUITE 9200, SEATTLE, WA, 98104-1158, US

(73) Assignee: Medlogics Device Corporation, Santa Rosa, CA (US)

(21) Appl. No.: 11/090,998

(22) Filed: Mar. 24, 2005

Related U.S. Application Data
(63) Continuation of application No. 10/196,296, filed on Jul. 15, 2002.
(60) Provisional application No. 60/323,071, filed on Sep. 19, 2001.
(60) Provisional application No. 60/333,523, filed on Nov. 28, 2001.
(60) Provisional application No. 60/364,083, filed on Mar. 15, 2002.

(51) Int. Cl. C25D 5/48; A61F 2/00

(52) U.S. Cl. 424/423; 205/220

(57) ABSTRACT

One embodiment of the invention is directed to a method comprising providing an electrochemical solution comprising metal ions and a bioactive material such as bioactive molecules, and then contacting the electrochemical solution and a substrate. A bioactive composite structure is formed on the substrate using an electrochemical process, where the bioactive composite structure includes a metal matrix and the bioactive material within the metal matrix.

### CROSS-REFERENCES TO RELATED APPLICATIONS

This non-provisional application is a continuation of U.S. Utility patent application Ser. No. 10/196,296, filed Jul. 15, 2002 which claims the benefit of the filing dates of the following U.S. Provisional Patent Applications: 60/323,071, filed Sep. 19, 2001, 60/333,523, filed Nov. 28, 2001, and 60/364,083 filed Mar. 15, 2002, the contents of which are herein incorporated by reference in their entirety for all purposes.

### FIELD OF THE INVENTION

The present invention relates generally to implantable medical devices. More specifically, it relates to methods of providing an electrochemical solution comprising metal ions and bioactive materials such as bioactive molecules and then contacting the electrochemical solution and a substrate. Still more particularly, it relates to providing a bioactive composite structure on a substrate using an electrochemical process.

### BACKGROUND OF THE INVENTION

In recent years, attempts have been made to produce biomimetic materials. Biomimetic materials are materials that imitate, copy, or learn from nature. Biomimetic materials can take many forms. For example, the surfaces of orthopedic implants can be porous to induce bony ingrowth from surrounding tissues.

Another form of a biomimetic material is one which releases a drug or other bioactive material. Drug release can accomplish many goals, one of which is to increase the biocompatibility of a material implanted in a patient.

Some stents can release drugs. A stent is a cylindrical device that is inserted into a body lumen to prevent blockage or collapse. Accordingly, stents are used to maintain lumen patency. Stents are predominantly used in the vascular system, e.g., the coronary, peripheral and cerebrovascular systems. The most common stents in use today are produced from stainless steel or nitinol. Stents are used in endovascular interventional procedures for diseases such as coronary artery disease, peripheral vascular disease, and cerebrovascular disease.

The hepatobiliary system is another place where stents are used. Indications for hepatobiliary stents include strictures and malignancy. Such stents are almost never long-term solutions. Permanent metal stents in the hepatobiliary system are placed for palliative treatment and only in patients who have less than six months to live.

A problem associated with stenting is the tendency for a lumen to re-narrow or “restenose” despite stenting. Research into the pathophysiology of “restenosis” in coronary artery disease has shown that there is smooth muscle cell proliferation and/or thrombosis shortly after a stent is placed within a vessel lumen. At present, the rate of restenosis, or failure, is 30-50% at six months, necessitating re-stenting and/or surgical correction. Over one million procedures are performed per year to open the coronary arteries, even after stents are placed within them.

Some stents under development are made biomimetic by releasing agents which target smooth muscle cells to prevent the process of restenosis. For example, some stents store drugs such as rapamycin or paclitaxel in a polymeric coating and then release them over time to combat restenosis. The polymeric coating releases the drugs via degradation of the polymer or diffusion into liquid (in this case the polymer is non-degradeable). Degradable and non-degradeable polymers such as polylactic acid, polyglycolic acid, and polymethylmethacrylate have been used in drug eluting stents.

There are a number of problems associated with using a polymeric material as a drug storage and release medium in stents and in medical devices in general. First, most polymeric coatings release bioactive materials relatively quickly and furthermore, it is difficult to predict the degradation kinetics of polymers. Consequently, it is difficult to predict how quickly a bioactive material in a polymeric medium will be released by the polymeric medium. If a drug releases from the medium too quickly or too slowly, the intended therapeutic effect may not be achieved. Second, in some cases, polymeric materials produce an inflammatory response. For example, a polymeric coating on a stent in a vessel can produce an inflammatory response on the vessel's walls, exacerbating restenosis. Third, adherence of a polymeric material to a substantially different substrate, such as a metallic substrate, e.g., a stent, is difficult. Mismatched properties such as different thermal expansion properties between the polymeric material and the underlying metallic stent body contribute to this difficulty. Inadequate bonding between the stent body and an overlying polymeric material may result in the separation of these two stent components over time, an undesirable property in an implanted medical device. Fourth, it is difficult to evenly coat a small metallic substrate with a polymeric material. As a small metallic object such as a stent is made smaller (e.g., less than 3 mm in diameter), it becomes more difficult to coat it evenly with a polymeric material. When the polymer is deposited, because it is viscous, it is difficult to evenly coat the object and faithfully replicate its form. Fifth, polymeric storage and release media are large and bulky relative to their bioactive material storage capacity. It would be desirable if the storage density of bioactive material storage medium could be increased so that a bioactive material could be released over a long period of time without increasing the bulk of the release media. Sixth, when delivering a bioactive material to a patient over a longer time period, particularly in an in-vivo environment, the bioactive material needs to be stabilized. Some polymeric materials may not provide for a stable storage environment for the bioactive material, in particular when liquid is able to seep into the polymeric material. Seventh, polymers, which by their nature have large pores, can protect micro-organisms in the interstices of the polymeric release medium, thus increasing the risk of infection. Eighth, polymer coatings currently under development contribute bulk but do not contribute to the major function of the stent, which is to prop open the body lumen. It would further be desirable if the storage medium for the bioactive material contributed to the mechanical strength of the object.

Sintered metallic structures could be used as an alternative to polymeric media. In a typical sintering process, small particles of metal are joined by an epoxy and then treated with heat and/or pressure to weld them together and to the substrate. A porous metallic structure has then been created. While effective in some instances, sintered metallic structures have relatively large pores. When a bioactive material is loaded into the pores of a sintered metallic structure, the larger pore size can cause the biologically active material to be released too quickly. Also, because a high temperature is used to form a sintered structure, a bioactive material including biologically active molecules must be loaded into the sintered structure after the porous structure is formed. This method is not only time consuming, it is also difficult to impregnate the pores of the sintered structure with biologically active molecules. Consequently, it is difficult to fully load the sintered structure with them. When impregnating a sintered structure, the bioactive molecules are in a carrier such as water. The surface tension of the carrier may preclude the biologically active molecules from thoroughly impregnating the sintered structure. As a result, the sintered structure may not be fully loaded with the biologically active molecules. As noted above, it would be desirable to have ability to increase the bioactive material storage capacity in a bioactive composite material so that, for example, the bioactive material can be released to a patient over a long period of time. Finally, because a liquid (blood, water, etc.) can enter into the pores of the material, the stability of the bioactive materials is limited.

Embodiments address the above problems and other problems, individually and collectively.

### SUMMARY OF THE INVENTION

Embodiments of the invention are directed to structures, methods, and devices that include a metallic matrix including a bioactive material (e.g., a drug). In embodiments of the invention, the bioactive material is contained within a metallic matrix. In some embodiments, the matrix can be crystalline and can have grain boundaries. Diffusion of the bioactive material can occur along the grain boundaries and crystallites of the metal. The bioactive material can be within, for example, nanometer and sub-nanometer sized voids in the metallic matrix. In embodiments of the invention, the bioactive material can be stored in a metallic matrix and can then be released from the metallic matrix. The bioactive material may diffuse through the metallic matrix or the metallic matrix could erode (actively and/or passively) to release the bioactive material over time. This can be done without using a polymeric storage and release medium for the bioactive material.

One embodiment of the invention is directed to a method comprising: (a) providing an electrochemical solution comprising metal ions and bioactive materials; (b) contacting the electrochemical solution and a substrate; and (c) forming a bioactive composite structure on the substrate using an electrochemical process, wherein the bioactive composite structure includes a metal matrix and the bioactive molecules within the metal matrix.

Another embodiment of the invention is directed to a bioactive composite structure comprising: (a) a metal matrix, wherein the metal matrix is formed using an electrochemical process; and (b) bioactive molecules within the metal matrix.

Other embodiments of the invention are directed to various devices such as medical devices that incorporate the bioactive composite structure or are wholly comprised of the bioactive composite structure.

Other embodiments of the invention are directed to methods of using the bioactive composite structure.

These and other embodiments of the invention are described in further detail with reference to the Figures and the Detailed Description.

Although medical devices such as stents are discussed in detail, it is understood that embodiments of the invention are not limited to stents or for that matter, to macroscopic devices. For example, embodiments of the invention could be used in any device or material, regardless of size and includes artificial hearts, plates, screws, mems (microelectromechanical systems), and nanoparticle based materials and systems, etc. Other examples of medical devices and materials according to embodiments of the invention are described below.

### BRIEF DESCRIPTION OF THE DRAWINGS

FIG. 1 shows a schematic illustration of a substrate and a bioactive composite structure on the substrate.

FIG. 2 shows a schematic illustration of a portion of a bioactive composite structure containing a bioactive material.

FIG. 3 shows a device including a bioactive composite structure in between a substrate and a topcoat.

FIGS. 4(a)-4(c) show a stent being placed into a coronary artery.

FIG. 5 shows a flowchart illustrating an exemplary method according to an embodiment of the invention.

FIG. 6 shows a graph showing drug elution profiles associated with Johnson and Johnson Bx velocity stents (stainless steel) with bioactive composite structures according to embodiments of the invention.

FIG. 7 shows a graph showing drug elution profiles associated with stents made with nitinol and bioactive composite structures according to embodiments of the invention.

### DETAILED DESCRIPTION

I. Definitions

Some terms that are used herein are described as follows.

The term “bioactive material” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a living or senescent cell, bacterium, virus, or part thereof. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active material. An example is a nanoparticle comprising a core with a drug and a coating on the core.

Bioactive materials may also include drugs such as chemical or biological compounds that can have a therapeutic effect on a biological organism. Bioactive materials include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological materials may include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases.

The term “electrochemical deposition” refers to both electrodeposition (electroplating) and electroless deposition (see method descriptions below).

The term “medical device” refers to an entity not produced in nature, which performs a function inside or on the surface of the human body. Medical devices include but are not limited to: biomaterials, drug delivery apparatuses, vascular conduits, stents, plates, screws, spinal cages, dental implants, dental fillings, braces, artificial joints, embolic devices, ventricular assist devices, artificial hearts, heart valves, venous filters, staples, clips, sutures, prosthetic meshes, pacemakers, pacemaker leads, defibrillators, neurostimulators, neurostimulator leads, and implantable or external sensors. Medical devices are not limited by size and include micromechanical systems and, nanomechanical systems which perform a function in or on the surface of the human body. Embodiments of the invention include such medical devices.

The term “implants” refers to a category of medical devices, which are implanted in a patient for some period of time. They can be diagnostic or therapeutic in nature, and long or short term.

The term “self-assembly” refers to a nanofabrication process to form a material or coating, which proceeds spontaneously from a set of ingredients. A common self-assembly process includes the self-assembly of an organic monolayer on a substrate. One example of this process is the binding of linear organic molecules to a substrate. Each molecule contains a thiol group (S-H moiety). The thiol group of each molecule couples to the gold surface while the other end of the molecule extends away from the gold surface. The process of electroless deposition, which continues spontaneously and auto-catalytically from a set of ingredients, may also be considered a self-assembly process.

The term “stents” refers to devices that are used to maintain patency of a body lumen or interstitial tract. There are two categories of stents; those which are balloon expandable (e.g., stainless steel) and those which are self expanding (e.g., nitinol). Stents are currently used in peripheral, coronary, and cerebrovascular vessels, the alimentary, hepatobiliary, and urologic systems, the liver parenchyma (e.g., porto-systemic shunts), and the spine (e.g., fusion cages). In the future, stents will be used in smaller vessels (currently stent diameters are limited to about 2 to 3 millimeters). For example, they will be used in the interstitium to create conduits between the ventricles of the heart and coronary arteries, or between coronary arteries and coronary veins. In the eye, stents are being developed for the Canal of Schlem to treat glaucoma.

The term “electroforming” refers to a process in which electrochemical deposition processes are performed on a sacrificial substrate. After the deposition process, the substrate is etched away, leaving a freestanding structure.

II. Methods of Manufacture

Embodiments of the invention include methods of manufacturing bioactive composite materials. In one embodiment, the method includes providing an electrochemical solution comprising metal ions and a bioactive material. The electrochemical solution may be an electroless deposition bath that is formed using metal salts, a solvent, and a reducing agent or a electrodeposition bath which is formed with a cathode (the substrate for deposition), an anode, and an electrolyte solution containing the metallic ions to be reduced. Complexing agents, stablizers, and buffers may also be present in the bath. After the electrochemical solution is formed, a substrate contacts the electrochemical solution. For example, the substrate may be immersed in a bath comprising the electrochemical solution.

Prior to contacting the electrochemical solution, the substrate can be prepared for the electrochemical process. In one preparation step, an anodic process is performed. In this process, the substrate is submerged in a hydrochloric acid bath. Current is passed through the solution, creating small pits in the substrate. Such pits promote adhesion. Also, a sensitizing agent and/or catalyst can be deposited on the substrate to assist in the creation of nucleation centers leading to the formation of the bioactive composite structure. Loosely adhered nucleation centers can also be removed from the surface of the substrate using, for example, a rinsing process.

After contacting the electrochemical solution, a bioactive composite structure is formed on the substrate using an electrochemical process. The electrochemical process may be an electrolytic or an electroless process (i.e. electro- or electroless deposition.) After forming the bioactive composite structure, the bioactive composite structure/substrate combination is removed from the bath containing the electrochemical solution.

After removing the bioactive composite structure/substrate combination from the bath, the combination may be further processed if desired. For example, in some embodiments, a topcoat may be formed on the bioactive composite structure. Additional details about the topcoat and other subsequent processing steps are described below.

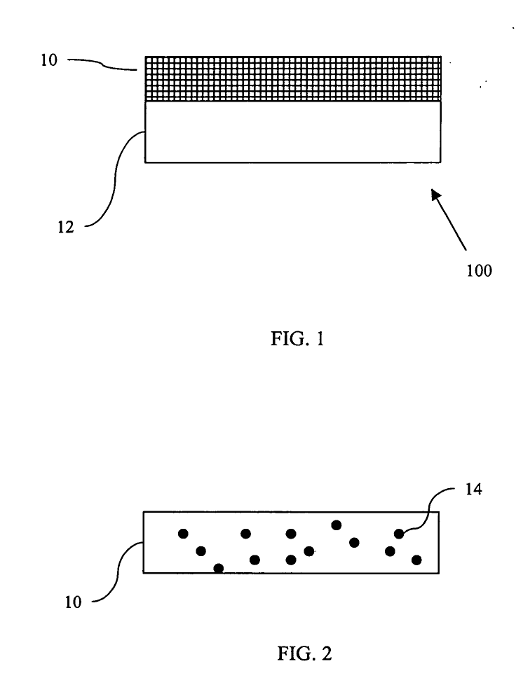

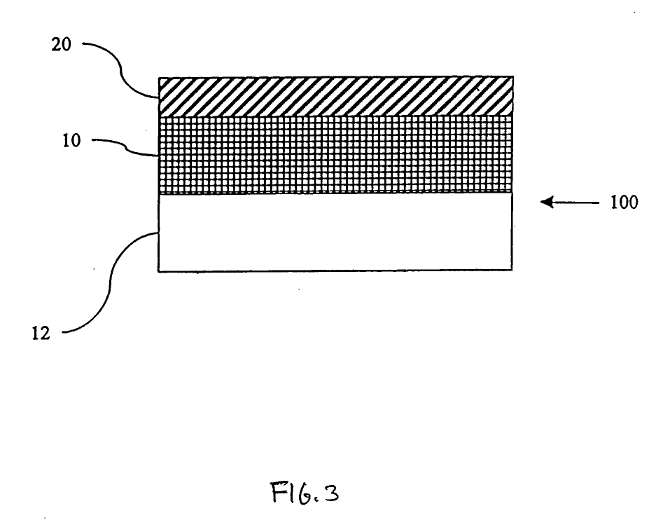

A device including a bioactive composite structure according to an embodiment of the invention is shown in FIGS. 1 and 2. The Figures depict a device 100 including a bioactive composite structure 101 including a metal matrix 10 and the bioactive material 14 within the metal matrix 10. The bioactive composite structure 101 is on a substrate 12. The proportion of bioactive material to the proportion of metal in a bioactive composite structure is high relative to the proportions of bioactive material that might be found in conventional bioactive composite structures, containing a metallic matrix.

Embodiments of the invention have a number of other advantages over conventional methods for forming bioactive composite structures. First, when bioactive materials are incorporated into a metallic matrix using an electrochemical process, the electrochemical process does not damage the bioactive material. Unlike high temperature processes for forming metallic matrices (e.g., sintering), embodiments of the invention can be performed at temperatures that do not harm bioactive materials (e.g., proteins). Second, in some embodiments of the invention, bioactive materials are more easily loaded into a metallic matrix than in conventional metallic matrices. For example, problems associated with impregnating a preformed metallic matrix with a solution comprising a carrier and a bioactive material are generally not present in embodiments of the invention. Consequently, the bioactive composite structures according to embodiments of the invention can have higher proportions of bioactive materials than conventional bioactive composite structures. Third, in some embodiments, the formed bioactive composite structure releases a bioactive material in a very localized area at specified times in an active and/or passive fashion over a period of months to years. The controlled and/or predictable release of the bioactive material can be achieved using embodiments of the invention. Fourth, when the bioactive composite material is in the form of a layer on a metallic substrate, the bioactive composite material and the metallic substrate can have similar properties. For example, the ductility and the modulae of elasticity of the bioactive composite material can be substantially the same as the underlying substrate. In another example, the metallic matrix of the bioactive composite structure and the substrate can both be metallic in embodiments of the invention. They can have similar thermal expansion coefficients, thus decreasing the likelihood that the two materials may separate due to thermal expansion differences. Fifth, the bioactive composite structures can be made uniform in composition and thickness in embodiments of the invention. If the bioactive composite structure is in the form of a layer on a metallic substrate with a complex shape, the layer can easily conform to the complex shape. Other advantages of embodiments of the invention are provided below.

A. Substrate Preparation

Any suitable substrate may be coated using embodiments of the invention. The substrate may be porous or solid, and may have a planar or non-planar surface (e.g., curved). The substrate could also be flexible or rigid. In some embodiments, the substrate may be a stent body, an implant body, a particle, a pellet, an electrode, etc.

The substrate may comprise any suitable material. For instance, the substrate may comprise a metal, ceramic, polymeric material, or a composite material. Illustratively, the substrate may comprise a metal such as stainless steel or nitinol (Ni—Ti alloy). Alternatively, the substrate may comprise a polymeric material including fluoropolymers such as polytetrafluoroethylene. In some embodiments, the substrate may comprise a sacrificial material. A sacrificial material is one that can be removed, for example, by etching, thereafter leaving a free-standing bioactive composite structure.

The substrate may be prepared in any suitable manner prior to forming a bioactive composite structure on it. For example, the substrate surface may be sensitized and/or catalyzed prior to performing an electroless deposition process (if the surface of the substrate is not itself autocatalytic). Metals such as Sn can be used as sensitizing agents. Many metals (e.g., Ni, Co, Cu, Ag, Au, Pd, Pt) are good auto catalysts. Palladium (Pd), platinum (Pt), and copper (Cu) are examples of “universal” nucleation center forming catalysts. In addition, many non-metals are good catalysts as well.

Before forming the bioactive composite structure, the substrate may also be rinsed and/or precleaned if desired. Any suitable rinsing or pre-cleaning liquid or gas could be used to remove impurities from the surface of the substrate before performing the electrochemical process. Also, in some embodiments involving electroless deposition, distilled water may be used to rinse the substrate after sensitizing and/or catalyzing, but before performing the electrochemical process in order to remove loosely attached molecules of the sensitizer and/or catalyst. In addition to, or in place of this, an anodic, or sometimes cathodic, cleaning process is used in some embodiments to produce pits which enhance adhesion.

B. Electrochemical Processes

In embodiments of the invention, an electrochemical deposition process is used to form the bioactive composite structure. Electrochemical deposition processes include electrolytic (electro) deposition and electroless deposition.

In embodiments of the invention, a bioactive material is incorporated into an electrochemical bath along with a source for metal ions. The bioactive material can include any of the particular materials mentioned above as well as other materials. For example, the bioactive material refers to any organic, inorganic, or living agent that is biologically active or relevant. The bioactive material could also comprise biologically active molecules such as drugs. In embodiments of the invention, the bioactive material may be soluble or insoluble in the electrochemical solution.

The bioactive material may also comprise particles (e.g., in the size range of 0.1 to about 10 microns). The particles may comprise the bioactive material in a crystallized form. Alternatively, the particles comprise a polymer, ceramic, or metal, which can store a bioactive material. The particles are preferably insoluble in the electrochemical solution. In this case, a particulate stabilizer such as a surfactant could be added to the electrochemical solution to improve the homogeneity of the particles in the solution.

Without being bound by theory, it is believed that when performing an electrochemical deposition process according to some embodiments, nanometer-sized crystallites (crystallized metal atoms) and the bioactive material “co-deposit”. At first, the process occurs on the surface of the substrate. Following the deposition of tens of nanometers of metal, the co-deposition occurs on the already deposited metal. Thus, the bioactive material and the metal atoms may deposit substantially simultaneously. When co-depositing metal atoms and the bioactive material, the bioactive material is incorporated into the metal matrix. These crystallites confine the bioactive material in the formed bioactive composite structure.

By co-depositing the bioactive material along with the metal, the concentration of the bioactive material in the bioactive composite structure is high. Moreover, the problems associated with impregnating porous structures with bioactive materials are not present in embodiments of the invention. In embodiments of the invention, the bioactive material substantially fills the voids in the metal matrix so that the loading of the bioactive material in the metal matrix is maximized.

As noted, electrochemical processes include electrolytic (electro) and electroless deposition processes. In electrolytic (electro) deposition, an anode and cathode are electrically coupled through an electrolyte. As current passes between the electrodes, metal is deposited on the cathode while it is either dissolved from the anode or originates from the electrolyte solution. Electrolytic deposition processes are well known in, for example, the metal plating industry and in the electronics industry.

An exemplary reaction sequence for the reduction of metal in an electrodeposition process is as follows:
M^{Z+}ₛₒₗᵤₜᵢₒₙ+ze→M_{lattice(electrode)}

In this equation, M is a metal atom, M^{Z+} is a metal ion with z charge units and e is an electron (carrying a unit charge). The reaction at the cathode is a (reduction) reaction and is the location where electrodeposition occurs. There is also an anode where oxidation takes place. To complete the circuit, an electrolyte solution is provided. The oxidation and reduction reactions occur in separate locations in the solution. In an electrolytic process, the substrate is a conductor as it serves as the cathode in the process. Specific electrolytic deposition conditions such as the current density, metal ion concentration, and bioactive material concentration can be determined by those of ordinary skill in the art.

Electroless deposition processes can also be used to form a bioactive composite structure. In an electroless deposition process, current does not pass through the solution. Rather, the oxidation and reduction processes both occur at the same “electrode” (i.e., on the substrate). It is for this reason that electroless deposition results in the deposition of a metal and an anodic product (e.g., nickel and nickel-phosphorus).

In an electroless deposition process, the fundamental reaction is:
M^{Z+}ₛₒₗᵤₜᵢₒₙ+R_{ed solution}→M_{lattice(catalytic surface)}+Oxₛₒₗᵤₜᵢₒₙ

In this equation, R is a reducing agent, which passes electrons to the substrate and the metal ions. Ox is the oxidized byproduct of the reaction. In an electroless process, electron transfer occurs at substrate reaction sites (initially the nucleation sites on the substrate; these then form into sites that are tens of nanometers in size). The reaction is first catalyzed by the substrate and is subsequently auto-catalyzed by the reduced metal as a metal matrix forms.

The electroless deposition solution can comprise metal ions and a bioactive material. Suitable bioactive materials are described above. The solvent that is used in the electroless deposition solution may include water so that the deposition solution is aqueous. Deposition conditions such as the pH, deposition time, bath constituents, and deposition temperature may be chosen by those of ordinary skill in the art.

Any suitable source of metal ions may be used in embodiments of the invention. The metal ions in the electrochemical solution can be derived from soluble metal salts before they are in the electrochemical solution. In solution, the ions forming the metal salts may dissociate from each other. Examples of suitable metal salts for nickel ions include nickel sulfate, nickel chloride, and nickel sulfamate. Examples of suitable metal salts for copper ions include cupric and cuprous salts such as cuprous chloride or sulfate. Examples of suitable metal salts for tin cations may include stannous chloride or stannous floroborate. Other suitable salts useful for depositing other metals are known in the electroless deposition art. Different types of salts can be used if a metal alloy matrix is to be formed.

The electrochemical solution may also include a reducing agent, complexing agents, stabilizers, and buffers. The reducing agent reduces the oxidation state of the metal ions in solution so that the metal ions deposit on the surface of the substrate as metal. Exemplary reducing compounds include boron compounds such as amine borane and phosphites such as sodium hypophosphite. Complexing agents are used to hold the metal in solution. Buffers and stabilizers are used to increase bath life and improve the stability of the bath. Buffers are used to control the pH of the electrochemical solution. Stabilizers can be used to keep the solution homogeneous. Exemplary stabilizers include lead, cadmium, copper ions, etc. Reducers, complexing agents, stabilizers and buffers are well known in the electroless deposition art and can be chosen by those of ordinary skill in the art.

Illustratively, a nickel-phosphorous alloy matrix can be electrolessly deposited on a substrate along with a bioactive material such as a drug. The substrate may need to be activated and/or catalyzed (using, e.g., by Sn and/or Pd) prior to metallizing. To produce this alloy matrix, a typical electroless deposition solution contains NiSO₄ (26 g/L), NaH₂PO₂ (26 g/L), Na-acetate (34 g/L) and malic acid (21 g/L). The solution may be in the form of a bath and may contain ions derived from the previously mentioned salts. A bioactive material is also in the bath. In this example, sodium hypophosphite is the reducing agent and nickel ions are reduced by the sodium hypophosphite. The temperature of the bath is from room temperature to 95° C. depending on desired plating time. The pH is generally from about 5 to about 7 (these processing conditions could be used in other embodiments). The substrate to be coated is then immersed in the solution and a bioactive composite structure can be formed on the substrate after a predetermined amount of time. The Ni ions in solution deposit onto the substrate as pure nickel (reduction reaction) along with nickel-phosphorous alloy (oxidation reaction); the bioactive material co-deposits along the crystallite and grain boundaries of the deposited metal matrix to form a bioactive composite structure. The bioactive material may co-deposit along with nickel atoms. Typically, the amount of phosphorous ranges from <1% to >25% (mole %) and can be varied by techniques known to those skilled in the art.

Although co-deposition of the metal atoms and the bioactive material is preferred, co-deposition is not necessary in some embodiments. For example, in other embodiments, a very thin metallic layer on the order of tens of nanometers can be formed on a substrate. A bioactive material is then either adsorbed, covalently bound, or deposited on top of the nanometer thick metallic layer. Additional metallic layers are subsequently added afterward. In between metallic layers, additional layers of bioactive material can be adsorbed, covalently bound, or deposited. This type of process produces a dense bioactive composite material.

The metallic matrix of the bioactive composite structure can include any suitable metal. The metal in the metallic matrix may be the same as or different from the substrate metal (if the substrate is metallic). The metallic matrix may include, for example, noble metals or transition metals. Suitable metals include nickel, copper, cobalt, palladium, platinum, chromium, iron, gold, and silver and alloys thereof. Examples of suitable nickel-based alloys include Ni—Cr, Ni—P, and Ni—B. Any of these or other metallic materials may be deposited using a suitable electrochemical process. Appropriate metal salts can be selected to provide appropriate metal ions in the electrochemical solution for the metal matrix that is to be formed.

The metallic matrix may also have voids in a crystal lattice. Typically, the average void size is less than about 1 micron. For example, in some embodiments, the average size of the voids in the metallic matrix may be less than about 100 angstroms (e.g., less than about 10 nanometers). The bioactive material can be incorporated into the voids of the metallic matrix.

In the formed bioactive composite material, the volume percent of the bioactive material is high. For example, in embodiments of the invention, the bioactive material can make up percentage of the bioactive composite structure. Preferably, the bioactive material can make up greater than about 10%, or greater than about 25% percent by volume of the bioactive material.

The bioactive composite structure may be in any suitable form. For example, the bioactive composite material may in the form of a layer on the substrate. The layer may have any suitable thickness. For example, the layer may have a thickness of less than about 100 microns in some embodiments (e.g., from about 0.5 to about 10 microns). In another example, the layer may have a thickness of greater than about 1 mm. In other embodiments, the bioactive composite structure need not be in the form of a layer. For example, the bioactive composite structure could be in the form of small particles in some embodiments.

Forming a bioactive composite structure using an electroless deposition process is advantageous. First, by using an electroless deposition process, the size of the crystallites and consequent percentage of bioactive material is controllable. Parameters such as the pH, temperature, and the constituents of the deposition bath can be adjusted by the person of ordinary skill in the art to alter the volume percentage of bioactive material in the formed metallic matrix. Second, using an electroless process, substrates having complex geometries can be evenly coated with a bioactive composite structure. As the solutions are aqueous in nature, viscous effects do not dominate in an electroless deposition process (as compared to coating polymeric substances which are viscous). Third, in an electroless deposition process, deposition conditions are mild, occurring at or near room temperature and at or near body physiologic pH. Bioactive materials are not damaged in the process of forming the bioactive composite material. Fourth, the methods according to embodiments of the invention are economical and scaleable, and are more cost-effective than other methods of forming bioactive composite structures.

C. Subsequent Processing

After the bioactive composite structure is formed, it may optionally be further processed in any suitable manner. For example, in some embodiments, a topcoat is formed on top of a bioactive composite structure. FIG. 3 illustrates a device 100 including a bioactive composite structure 10 in the form of a layer in between a substrate 12 and a topcoat 20.

The topcoat can include any suitable material and may be in any suitable form. It can be amorphous or crystalline, and may include a metal, polymer, ceramic, etc. The topcoat may also be porous or solid (continuous).

The topcoat can be deposited using any suitable process. For example, the above-described processes (e.g., electro- and electroless deposition) could be used to form the topcoat or another process may be used to form the topcoat. Alternatively, the topcoat could be formed by processes such as dip coating, spray coating, vapor deposition, etc.

The thickness of the topcoat may vary in embodiments of the invention. For example, in some embodiments, the topcoat may have a thickness greater than about 100 microns. Of course, the thickness of the topcoat can depend on the end use for the device being formed.

In embodiments of the invention, the topcoat may be the only layer on the bioactive composite structure. In other embodiments, any number of suitable topcoat layers may be added to the bioactive composite structure. For example, it is possible that tens to hundreds of individual layers could be formed on the bioactive composite structure (some or all of these layers may be bioactive).

In some embodiments, the topcoat can improve the properties of the bioactive composite structure. For example, the topcoat may include a membrane (e.g., collagen type 4) that is covalently bound to the bioactive composite structure. The topcoat's function can be to induce endothelial attachment to the surface of the bioactive composite structure, while the bioactive material in the bioactive composite structure diffuses from below the topcoat. In another embodiment, a growth factor such as endothelial growth factor (EGF) or vascular endothelial growth factor (VEGF) is present in a topcoat that is on the bioactive composite structure. The growth factor is released from the topcoat to induce endothelial growth while the bioactive composite structure releases an inhibitor of smooth muscle cell growth.

In yet other embodiments, the topcoat can improve the radio-opacity of a medical device which includes the bioactive composite structure, while the underlying bioactive composite structure releases molecules to perform another function. For example, drugs can be released from the bioactive composite structure to prevent smooth muscle cell overgrowth, while a topcoat on the bioactive composite structure improves the radio-opacity of the formed medical device. Illustratively, a topcoat comprising Ni—Cr (nickel chromium) and/or gold can be deposited on top of a bioactive composite structure comprising Ni—P to enhance the radio-opacity of a device incorporating the bioactive composite structure. Underneath the topcoat, a smooth muscle cell inhibitor such as sirolimus is released over a 30-60 day time period from the bioactive composite structure.

The topcoat can also be used to alter the release kinetics of the bioactive material in the underlying bioactive composite structure. For example, an electroless nickel-chrome, nickel-phosphorous, or cobalt-chrome coating without bioactive material can serve as a topcoat. This would require the bioactive material to travel through an additional layer of material before entering the surrounding environment, thereby delaying the release of bioactive material. The release kinetics of the formed medical device can be adjusted in this manner.

Alternatively, the topcoat comprises a polymeric material (or other material). In this case, a bioactive material that is the same or different than the bioactive material in the bioactive composite structure may be included in the topcoat. For example, when the topcoat comprises a polymeric storage and release medium, the bioactive material therein can release quickly (e.g., days) from the topcoat, while the material in the bioactive composite structure is released over a period of months to years. In this embodiment, the medical device that is formed may include the combination of a topcoat comprising a polymeric storage and release medium, and a metallic storage and release medium.

Suitable polymers in the topcoat are preferably biocompatible (i.e., they do not elicit any negative tissue reaction) and can be degradable. Such polymers may include lactone-based polyesters or copolyesters, for example, polylactide, polycaprolacton-glycolide, polyorthoesters, polyanhydrides; poly-aminoacids; polysaccharides; polyphosphazenes; and poly (ether-ester) copolymers.

Nonabsorbable biocompatible polymers may also be used in the topcoat. Such polymers may include, for example, polydimethylsiloxane; poly(ethylene-vinylacetate); acrylate based polymers or copolymers, e.g., poly(hydroxyethyl methylmethacrylate); fluorinated polymers such as polytetrafluoroethylene; and cellulose esters.

In yet other embodiments, the topcoat that is on the bioactive composite structure can be a self-assembled monolayer (SAM). The thickness of the self-assembled monolayer may be less than 1 nanometer (i.e., a molecular monolayer) in some embodiments. In one example, a thiol based monolayer can be adsorbed on a nickel matrix of a bioactive composite structure through the thiol functional group and can self-assemble on the nickel matrix. The introduction of the self-assembled monolayer can permit different surface ligands to be used with the bioactive composite structure. That is, various ligands or moieties can be attached to the ends of the molecules in the monolayer that extend away from the bioactive composite structure.

In another embodiment, after forming the bioactive composite structure on a substrate, the substrate can be removed. This could be done to electroform a free-standing bioactive composite structure. For example, as noted above, when forming a medical device, a bioactive composite structure can be formed on a substrate. However, instead of leaving the substrate in the final medical device, the substrate may be etched to remove it from the formed bioactive composite structure. For example, the substrate may comprise an etchable material. Etchable materials include metals such as aluminum or copper or polymeric substances.

The substrate is a sacrificial substrate and can be used as a mandrel for forming a free-standing bioactive composite structure. After etching the substrate, a free-standing bioactive composite structure is formed. Stents, for example, can be formed in this manner. Details regarding the formation of stents using sacrificial substrates are found in U.S. Pat. No. 6,019,784. This U.S. Patent is herein incorporated by reference in its entirety.

The free-standing bioactive composite structure may have dimension on the order of nanometers (e.g., nanoparticles) to meters. For example, the thickness of the free-standing bioactive composite structure may be less than about 1 mm thick. As in other embodiments, a topcoat could be formed on a free-standing bioactive composite structure.

III. Releasing Bioactive Material from a Bioactive Composite Structure

The bioactive composite structures according to embodiments of the invention can be present in medical devices that are used in vivo. They can be implanted in the body of a patient when used, or could be used external to the body of a patient. In such medical devices, the long term release of a bioactive material from the bioactive composite material is desirable in some instances.

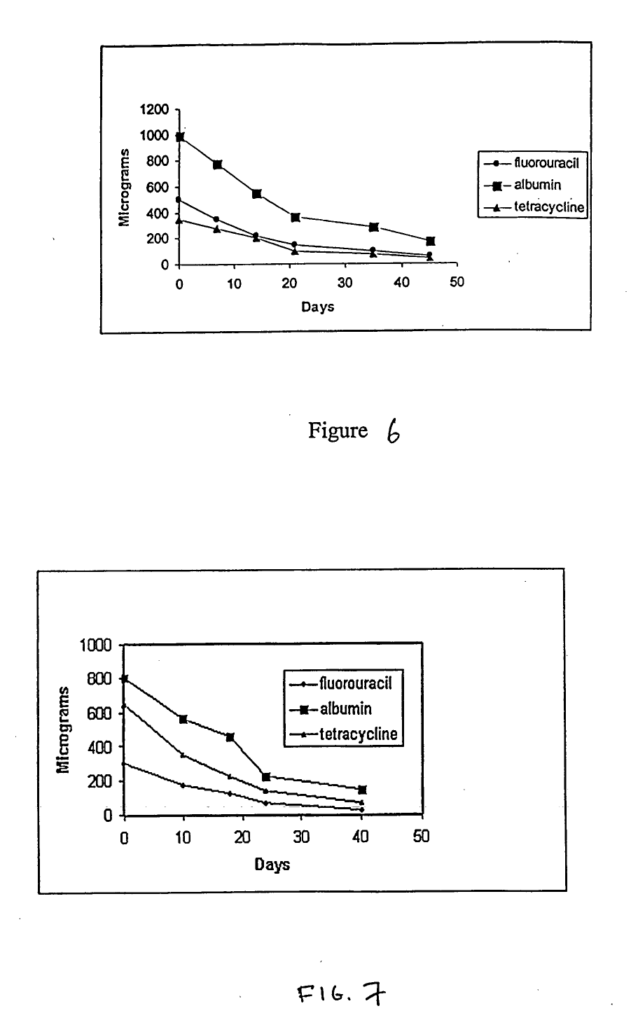

In some embodiments, the bioactive material can diffuse from the metallic matrix in the bioactive composite structure. FIGS. 6 and 7 (described in further detail below) show the results of experiments using embodiments of the invention. As shown in FIGS. 6 and 7, in embodiments of the invention, drugs can be released over long periods of time (e.g., greater than about 10 or about 20 days). Again, without being bound by theory, the release mechanisms in the examples shown in FIGS. 6 and 7 are indicative of simple diffusion. The bioactive material diffuses through the metallic matrix, that is, between individual crystallites and grain boundaries. The bioactive material exchanges places with the components of the metallic film and then diffuses into liquid at the interface of the metallic film and liquid.

Alternatively, the metallic matrix of the bioactive composite structure can erode to release the bioactive material in it. For example, the metallic matrix can be susceptible to electrolytic corrosion. The metallic matrix of the bioactive composite structure can serve as an anode, which results in corrosion of the metallic matrix when current is passed through a circuit which includes the composite structure as an anode. As a result of the corrosion process, the bioactive material is liberated from the metallic matrix. This is useful both in vivo and in vitro. By using a corrosion process, small, controllable quantities of a bioactive material (e.g., a drug or DNA) can be released in a highly localized regions at specified times within a patient or within a diagnostic assay.

Corrosion can occur actively or passively. In an active corrosion process, current is actively applied to the bioactive composite structure using an external power source to corrode the metallic matrix. In a passive corrosion process, the oxidation of the matrix metal of the bioactive composite material can be caused by the difference between the electrical potential of the metallic matrix and an adjacent metal or solution. For example, galvanic corrosion is caused when two metal pieces, in electrical contact with each other, or two adjacent metal areas are at different electrochemical potential. The two metal parts will constitute a galvanic cell, in which the metal part with the lowest electrochemical potential (i.e., the more active metal) will corrode.

In another embodiment, mechanical energy such as ultrasonic energy is applied to the bioactive composite structure. The mechanical energy hastens the rate of diffusion of the bioactive material from the bioactive composite structure. In this embodiment, the metallic matrix may or may not erode. In the case of a stent or other implanted medical device, ultrasonic energy may be applied non-invasively to a patient so that the release of the bioactive material from the stent can occur at a desired time. For example, the application of ultrasonic energy can be, for instance, days, weeks, or months after the stent is implanted.

IV. Medical Devices

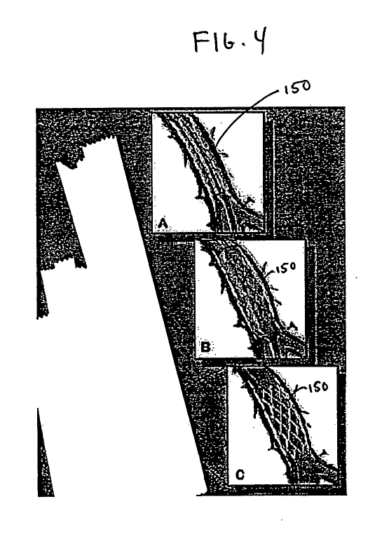

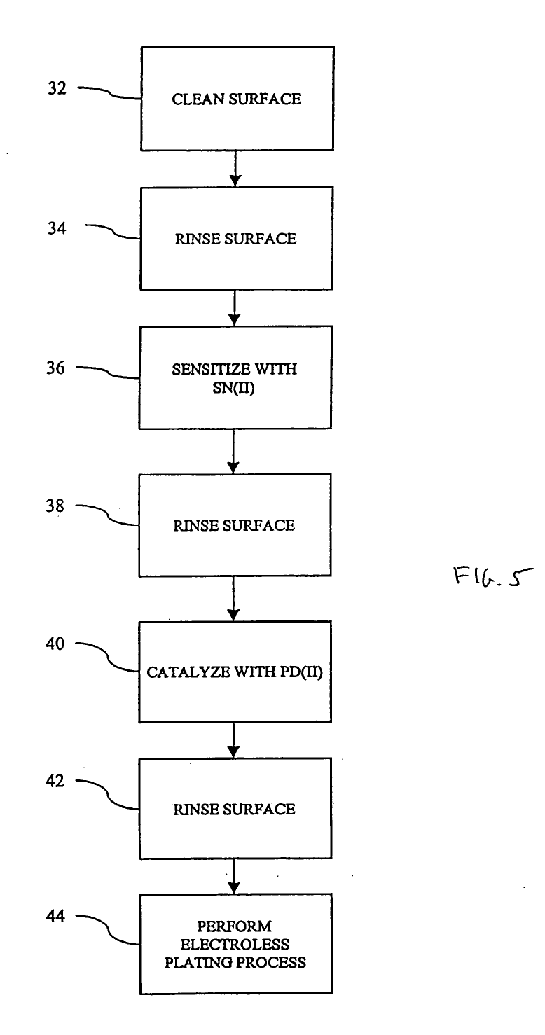

Embodiments of the invention include any suitable medical device incorporating the bioactive composite structure. For example, medical devices according to embodiments of the invention include stents, orthopedic implants, cardiovascular implants, electrodes, sensors, drug delivery capsules, surgical clips, micromechanical systems, and nanomechanical systems. A schematic drawing of a stent 150 in an artery is shown in FIGS. 4(a)-4(c).

In other embodiments, the bioactive composite structures are applied to blood or tissue contacting medical devices, which are dependent on endothelialization of the implant surfaces for biocompatibility. These devices include ventricular assist devices (VADs), total artificial hearts (TAHs), and heart valves. In comparison to stents, which have discontinuous surfaces (e.g., wire meshes with windows), these devices have continuous surfaces. They rely on cell seeding from the bloodstream. Accordingly, the bioactive composite structures can comprise growth factors. The bioactive composite structures provide an attachment surface that could facilitate the attachment and subsequent growth processes of endothelial cells on the surface. Such growth factors include any of a host of integrins, selecting, growth factors, and peptides, which can assist and hasten cell migration and adhesion.

The bioactive composite structures could also be used in drug release devices such as ingestible pills or devices capable of traveling in the bloodstream. These devices can take the form of a sphere, square or cylinder of sufficient size to fit into a body cavity. They can be placed in the human body transcutaneously or orally. Subsequent release occurs from the metallic matrix by one of the methods described above. This type of drug storage and delivery system can be produced in combination with other delivery vehicles such as biodegradeable polymers.

In another embodiment, the bioactive composite material may be present in wells or channels in a microchip-type device. The bioactive composite material in the wells or channels can be covered with a topcoat that is erodable. For example, the metallic matrix of the bioactive composite structure may comprise nickel or a nickel alloy, while the topcoat comprises gold. Electrical current is selectively applied to the gold topcoat, thereby causing it to erode. As a result of the erosion process, the bioactive material is free to diffuse out of each well or channel. Alternatively, the release of bioactive material from each well or channel can be induced by an electrical current. Passive corrosion can be induced by a bimetallic EMF (electromotive force) created by the combination of two metals. Active release can be induced by current induced erosion of the metallic matrix. In both cases, the amount of current applied to the metallic matrix can be directly proportion to the amount of released bioactive material. This design reduces the complexity of such systems compared to current designs.

Aside from use in therapeutic medical devices, the bioactive composite structure can be used in diagnostic devices and bioassays where a precise quantity of bioactive material is required in a spatially and/or temporally controlled fashion. They can be used in the drug discovery process. Bioassays for drug discovery are increasing in complexity and in many cases utilize live cells for bioassays. Modem surface technologies make it possible to study the effects of local chemical gradients in the study of cell response as well as local environmental alterations in cell culture, such as pH. Utilizing embodiments of the invention, dynamic release of bioactive materials at specific places at specific times and in controlled quantities could be used in diagnostic devices and bioassays.

In one embodiment, a bioactive composite structure is formed underneath the surface on which cells are cultured. The bioactive composite structure can be in the form of a pattern with varying concentrations of bioactive materials or in a layer containing one concentration of molecule. When appropriate, the matrix of the bioactive composite structure is dissolved via electrolytic corrosion and the bioactive material is released almost instantaneously into the environment surrounding the cells of interest. The amount of applied current determines the amount of bioactive material released.

This type of technology is meant to mimic the in vivo environment and can be used to study the molecular effects of specific molecules on cells at specific times identified with other biological assays. For example, the affect of molecule X on the cell cycle during G1 or G2, etc. where G1 and G2 are measured with a well-known assay such as a fluorescence assay.

### EXAMPLE I

Six bioactive composite structures were formed. Each bioactive composite structure comprised a nickel-phosphorous metallic matrix formed on a metallic substrate using an electroless deposition process. The substrates used were foils. Three substrates comprised medical grade 316 L stainless steel and three substrates comprised nitinol. fluorouracil, tetracycline, and albumin were respectively co-deposited with the nickel-phosphorous on the stainless steel and nitinol substrates.

Each substrate was first prepared using process steps show in FIG. 4. First, the surface of the substrate is cleaned (step 32). Then, the substrate surface is rinsed with distilled water (step 34). After rinsing, the surface of a substrate is sensitized with Sn(II) (step 36). A solution of 0.1 g/L of stannous chloride may be used as a sensitizing solution. After depositing Sn(II) on the surface of the substrate, the substrate is again rinsed with distilled water (step 38) in a second rinse step. Then, a Pd(II) catalyst is deposited on the surface of the substrate. A solution of 0.1 g/L palladium chloride may be used as a catalyzing solution (step 40). The surface of the substrate is again rinsed in a third rinsing step (step 42). Distilled water may be used as the rinsing fluid. After the third rinsing step, the substrate is catalyzed and is ready for electroless deposition. Three stainless steel and three nitinol substrates were prepared using the above described catalyzing process.

Three different electroless plating baths were made. The three different baths were the same, except that the bioactive material was different in each bath. Bath 1 contained 5-fluorouracil, Bath 2 contained tetracycline, and Bath 3 contained albumin. Each bath was at ambient pressure, at a pH of about 7, and at a temperature of about 40° C.

TABLE 1

| Ingredient | Concentration |
|---|---|
| Nickel Sulfamate | 29 g/L |
| Sodium Hypophosphite | 17 g/L |
| Sodium Succinate | 15 g/L |
| Succinic Acid | 1.3 g/L |
| Bioactive material: 5- | 0.25 g/L (Bath 1), 0.25 g/L |
| fluorouracil (Bath 1), | (Bath 2), and 100 μg/mL |
| tetracycline (Bath 2), and | (Bath 3) |
| albumin (Bath 3) | |

Six bioactive composite structures in the form of layers were respectively formed on the substrates (3 stainless steel substrates and 3 nitinol substrates) using electroless deposition (step 44). In general, the time in the bath determines the thickness of the bioactive composite structure. Each substrate was immersed in a bath for about 10 minutes to yield a layer about 4 microns thick. The concentration of the bioactive material in the bath determines the concentration of the bioactive material in the coating. For example, when albumin was used as a bioactive material, the concentration in the coating was 1:10 w/w albumin:metal with 100 μg/ml concentration of albumin in the starting bath.

For each bioactive composite structure, the weight proportion of the bioactive material to the metallic matrix material is listed in Table 2.

The weight proportions of the bioactive materials to the metallic matrices for each bioactive composite material were determined as follows. For each bioactive composite structure/substrate combination, pre- and post-deposition dry weights were measured. After they were formed, each bioactive composite structure/substrate combination was then placed in an electrolytic bath, with the bioactive composite structure being made the anode of an electrolytic circuit. With current introduced into the bath, the metallic matrix of the bioactive composite structure was corroded and passed from the substrate into the electrolytic bath. The amount of the bioactive material in the bath was then optically measured with the use of a spectrophotometer. The numbers below in Table 2 represent the weightₓ/weight_{Ni—P}, wherein the x represents the bioactive material and Ni—P is the electrochemically deposited metal matrix. As shown by the results in Table 2, the concentration of bioactive material to metal is high in each case.

TABLE 2
W/W concentration of bioactive material to deposited
Ni—P matrix on nitinol and 316L substrates

| | Fluorouracil | Tetracycline | Albumin |
|---|---|---|---|
| Nitinol | 0.100 mg/3 mg | 0.3 mg/4 mg | 0.5 mg/4.8 mg |
| 316L Stainless | 0.4 mg/3 mg | 0.5 mg/4 mg | 0.4 mg/4 mg |
| Steel | | | |

### EXAMPLE 2

Coated stents were formed using the same basic electroless deposition procedure in Example 1. However, in this example, instead of foil substrates, Johnson and Johnson Bx velocity stents (stainless steel) and Johnson and Johnson Smart stents (nitinol) were used as substrates. Bioactive composite structures in the form of layers were formed on the stents.

FIG. 6 shows a graph of the drug elution profiles when Johnson and Johnson Bx Velocity stents (316L stainless steel) were used as substrates. FIG. 7 shows a graph of the drug elution profiles when Johnson and Johnson Smart stents (nitinol) were used as substrates. The amounts on the y-axis of the graphs represent the amount of bioactive material remaining on the stent after elution into a physiologic saline solution.

A similar anodization process as was used in the stent examples as was again applied to the foil substrates. After coating, the coated stent was placed in a physiologic saline solution and the solution changed daily. On the indicated days, the stent coatings were anodized. The amount of bioactive material released in each case was determined using a spectrophotometric assay.

As can be seen in FIGS. 6 and 7, molecules are released from embodiments of the invention over long periods of time. Appreciable amounts of drugs such as fluorouracil, albumin, and tetracycline were released over 40 days. No appreciable corrosion of the coating was observed.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention.

All U.S. Patent Applications, Patents and references mentioned above are herein incorporated by reference in their entirety for all purposes.

1. A process for forming a bioactive material delivery device comprising the steps of:
providing a substrate; and
electroplating onto said substrate a porous layer having voids.

2. The process of claim 1, wherein said electroplating step comprises electroplating a material that is substantially free of the bioactive material to be delivered.

3. The process of claim 2, wherein the bioactive material is one or more drugs.

4. The process of claim 3, further comprising after said electroplating step, the step of loading said one or more drugs into said voids.

5. The process of claim 4, wherein after the step of loading one or drugs into said voids, further comprising the step of forming a topcoat over said drug loaded layer.

6. The process of claim 5, wherein said step of forming a topcoat comprises applying a polymer over said drug loaded layer.

7. The process of claim 1, wherein one of the conditions for controlling the deposition of the porous layer onto the substrate is by control of current density during the electroplating step.

8. The process of claim 1, wherein said bioactive material delivery device is a stent.

9. The process of claim 1, wherein said electroplating step comprises electroplating a metal selected from the group consisting of gold, nickel, silver, copper, palladium, platinum, cobalt, chromium, iron, and alloys thereof.

10. The process of claim 1, wherein between said step of providing a substrate and said step of electroplating said porous layer, further comprising the step of electroplating a solid layer onto said substrate.

11. The process of claim 1, further comprising the step of forming a topcoat over said porous layer.

12. The process of claim 11, wherein said step of forming a topcoat comprises applying a polymer over said porous layer.

13. The process of claim 1, wherein the bioactive material to be delivered is co-deposited during the electroplating step.

14. The process of claim 1, wherein said voids have an average void size of less than about 1 micron.

15. The process of claim 14, wherein said average void size is less than about 10 nanometers.

16. A process for forming drug delivery stent comprising:
providing a mandrel;
coating said mandrel with a resist;
exposing portions of said resist to a light pattern so as to form a stent pattern on said mandrel in said resist;
electroplating a stent on said mandrel;
electroplating a porous layer having voids on said stent; and
removing said resist and said mandrel.

17. The process of claim 16, wherein said electroplating step comprises electroplating a material that is substantially free of the drug to be delivered.

18. The process of claim 17, further comprising controlling current density during the electroplating step.

19. A process for forming a drug delivery stent comprising:
providing a prefabricated stent; and
electroplating a porous layer having voids on said stent.

20. The process of claim 19, wherein said electroplating step comprises electroplating a material that is substantially free of the drug to be delivered.

21. The process of claim 20, further comprising controlling current density during the electroplating step.
